(19) United States
(12) Patent Application Publication
(10) Pub. No.: US 2011/0054155 A1
(43) Pub. Date: Mar. 3, 2011

(54) Novel Environmentally Friendly Technology for Production of Ginsenoside Re

(76) Inventor: Zhongqing Lu, (US)

(21) Appl. No.: 12/549,444

(22) Filed: Aug. 28, 2009

(51) Int. Cl. C07J 43/00

(52) U.S. Cl. 536/5

(57) ABSTRACT

This invention relates to a technology used for a fast separation of Ginsenoside Re directly from other ginsensosides with similar molecular structures. In a conventional method, multiple steps and complex organic mobile phases are required to obtain a highly purified Ginsenoside Re compound, which are time consuming and organic solvent consumption, an obstacle for large scale industrial production. In our new technology, a quick separation for a mixture of two compounds with similar molecular structure. Ginsenoside Re and Ginsenoside Rg1 was demonstrated through a Surpass™ PolyGin-Re column 2×250 mm, in which only methanol was used for elution, no acids or bases were needed, which could be a Green technology for Ginsenoside Re production in the future.

### REFERENCES CITED IN

Note: No US and Foreign patent Documents are cited. All of the references cited herein are journal articles
T. O. Chen, Panax (Ginseng) is not a panacea. Arch. Int. Med.; 160: 3329-30, 2000
P A G M De Smet, Herbal remedies, N. Engl. J. Med.; 347: 2046-56, 2002
Scott, G. I., Colligan, P. B., Ren, B. H., Ren, J., Ginsenosides Rb1 and Re1 decrease cardiac concentration in adult rat ventricular myocytes: role of nitric oxide. Br. J. Pharmacol. 134, 1159-1165, 2001
J. T. Xie, S. R. Mehendale, X. M. Li, R. Quigg, X. Y. Wane, C. Z. Wang, J. A. Wu, H. H. Aung, P. A. Rue, G. I. Bell, C. S. Yuan, Anti-diabetic effect of ginsenoside Re in ob/ob mice. Biochimica et Biophysica Acta 1740, 319-325, 2005
T. Furukawa, C. Bai, A. Kaihara, E. Ozaki, T. Kawano, Y. Nakaya, M. Awais, M. Sato, Y. Umezawa, and J. Kurokawa, Ginsenoside Re, A Main Phytosterol of Panax ginseng, Activates Cardiac Potassium Channels via a Nongenomic Pathway of Sex Hormones. Molecular Pharmacology 70: 1916-1924, 2006
H. Zhang, Q. Zhou, X. Li, W. Zhao, Y. wang, H. Liu, N. Li, Ginsenoside Re promotes human sperm capacitation through nitric oxide-dependent pathway, Molecular Reproduction and Development, Vol. 74 (4), 497-501, 2006
Y. Wane, A. Zima, X. Ji, R. Pabbidi, L. A. Blatter, and S. L. Lipsius, Ginsenoside Re suppresses electromechanical alternans in cat and human cardiomyocytes, Am J. Physiol Heart Circ. Physiol 295: 851-859, 2008
Cai Xiong. Liu Zhongqiu, Wang Pei-xun, Liu Liang, Study on purification process of ginsenosides with macroreticular resin. Chinese Traditional Patent Medicine, Vol. 23 (9), 631-634, 2001
Li Honggang, He Kejiang, Han Wei, Luan Hongwei, Yang Ling. A purification method for total ginsenosides by resins, Chinese Archives of Traditional Chinese Medicine, Vol. 23 (4), 707-710, 2005
Chen Jing, Xu Qun, Jeff Rohrer, Li Lang, LC-GC Asia Pacific, volume 10 (4), 2007

### BACKGROUND OF THE INVENTION

Ginseng root has been used as an important intergradient in traditional Chinese medicine to treat a variety of ailments for more than 2000 years. Now, ginseng root is a popular herbal medicine in the United States [T. O. Chen, Panax (Ginseng) is not a panacea. Arch Int Med; 160: 3329-30, 2000], [P A G M De Smet, Herbal remedies, N. Engl. J. Med.; 347: 2046-56, 2002] Among over thirty ginsenosides, the active medicinal constituents found in ginseng extracts recently, Ginsenoside Re has shown its unique pharmacological properties. It was not only shown to stimulate the activity of nitric oxide synthase (NOS) significantly [Scott, G. I., Culligan, P. B., Ren, B. H., Ren, J., 2001. Ginsenosides Rb1 and Re1 decrease cardiac concentration in adult rat ventricular myocytes: role of nitric oxide. Br. J. Pharmacol. 134, 1159-1165], but also some functionality in anti-diabetic activities [J. T. Xie, S. R. Mehendale, X. M. Li, R. Quigg, X. Y. Wang, C. Z. Wang, J. A. Wu, H. H. Aung, P. A. Rue, G. I. Bell, C. S. Yuan, Anti-diabetic effect of ginsenoside Re in ob/ob mice. Biochimica et Biophysica Acta 1740, 319-325, 2005], activation of cardiac potassium channels [T. Furukawa, C. Bai, A. Kaihara, E. Ozaki, T. Kawano, Y. Nakaya, M. Awais. M. Sato, Y. Umezawa, and J. Kurokawa, Ginsenoside Re, A Main Phytosterol of Panax ginseng, Activates Cardiac Potassium Channels via a Nongenomic Pathway of Sex Hormones. Molecular Pharmacology 70: 1916-1924, 2006], promotion of human sperm capacitation [H. Zhang, Q. Zhou, X. Li, W. Zhao, Y. Wang, H. Liu, N. Li. Ginsenoside Re promotes human sperm capacitation through nitric oxide-dependent pathway, Molecular Reproduction and Development, Vol. 74 (4), 497-501, 2006], suppression of electromechanical alternans in cat and human cardiomyocytes [Y. Wang, A. Zima, X. Ji, R. Pabbidi, L. A. Blatter, and S. L. Lipsius, Ginsenoside Re suppresses electromechanical alternans in cat and human cardiomyocytes, Am J. Physiol Heart Circ. Physiol 295: 851-859, 2008].

In order to get a complete and pure medicinal cure using the individual constituent in Ginseng herbs, separation is crucial. A traditional purifying process for total ginsenosides is complicated, involving the use of macroreticular resins such as D101 and AB-8 adsorption resin and D280 ion exchange resin [Cai Xiong, Liu Zhongqiu, Wang Pei-Nun, Liu Liang, Study on purification process of ginsenosides with macroreticular resin. Chinese Traditional Patent Medicine Vol. 23 (9), 631-634, 2001] [Li Honggang, He Kejiang, Han Wei, Luan Hongwei, Yang Ling, A purification method for total ginsenosides by resins, Chinese Archives of Traditional Chinese Medicine, Vol. 23 (4), 707-710, 2005].

To obtain the pure ginsenoside compound from the mixture of ginsenosides above, further complicated purification is needed, usually including repeated separating steps with C18 columns and the use of delicate organic solvents due to the molecular structure similarity between ginsenosides. Apparently, the conventional technology currently used is inefficient, expensive, and not environmentally-friendly. Here a novel and unique process technology was discovered: Ginsenoside Re is separated from Ginsenoside. Rg1 swiftly in a single step with simple isocratic mobile phase, methanol only. This process technology can be used for expedient purification of Ginsenoside Re from ginseng leaf extracts directly in a single step, avoiding tremendous acid and base resources used by resin regeneration in conventional purifying processes. In addition, the purification time can be reduced dramatically, reduced from several days to a few hours for production of highly-purified products.

### SUMMARY OF THE INVENTION

It is an object of the present invention to overcome the aforesaid purifying deficiencies for Ginsenoside compounds in the prior art

It is an object of the present invention to use MIP resins packed columns to isolate Ginsenoside Re directly from Ginsenosides Rg1, Rb1

It is an object of the present invention to use MIP resins packed columns to analyze and isolate Ginsenoside Re in ginseng leaf extracts.

### LEGENDS OF THE CAPTIONS

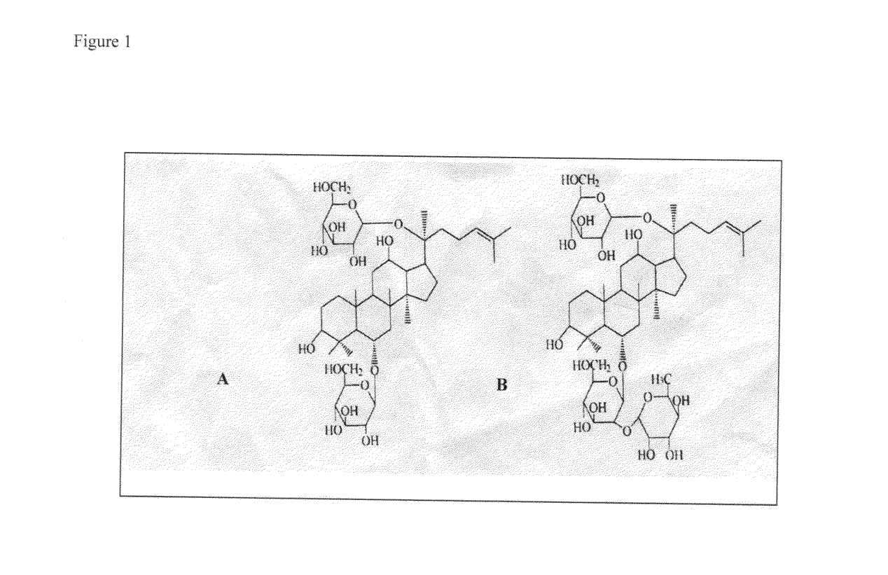

FIG. 1 Chemical structures of Ginsenosides Rg1 (a) and Re (b)

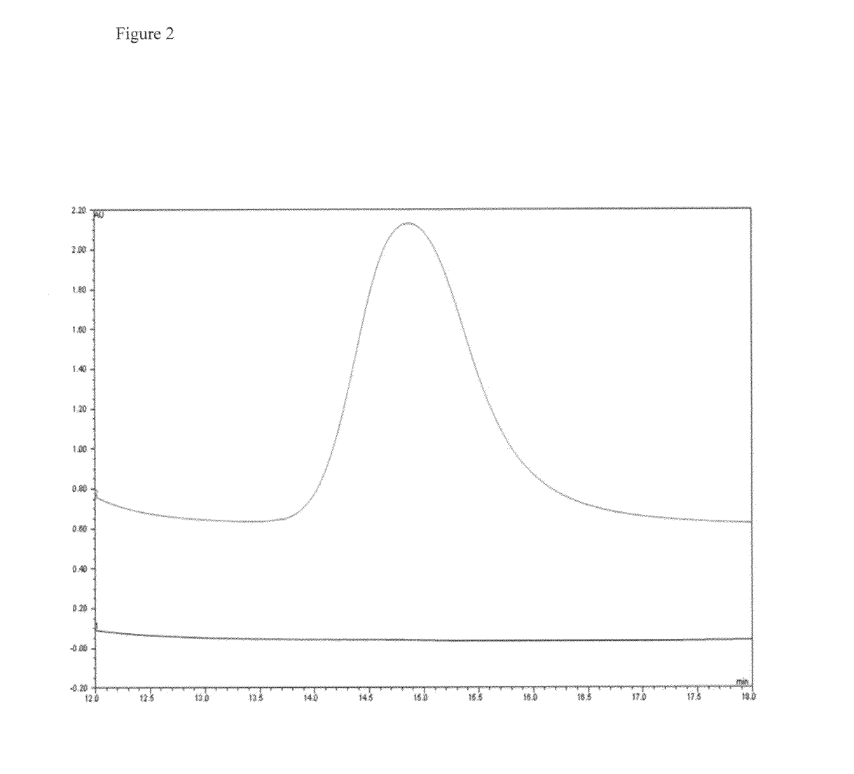

FIG. 2 Chromatogram for a mixture of Ginsenosides Rg1 and Re

### DETAILED DESCRIPTION OF THE INVENTION

The following detailed description are provided for purposes of describing and illustrating presently preferred embodiments of the invention only, and are not intended to limit the scope of the invention in any way.

### (a) Ginsenoside Re Imprinted Resins (Gin-Re MIP)

Due to the advantages of reusability and low cost compared to bio-system-like antibody, molecularly imprinted polymers (MIPs) have been widely used in various areas including chromatography stationary phase for separation purpose, solid phase extraction, antibody and receptor mimics, sensor strategies, and catalysis studies, even applications for the recognition of proteins. Among them, commercial SPE cartridge was used for sample preparation, especially for biological specimens, e.g. to adsorb ginsenosides in urine or blood samples first, followed by wash-off for HPLC analysis. Because the similar molecular structures for Ginsenoside compounds, a very complicated condition was required for use of an HPLC C18 column, eventually still not a satisfactory resolution was achieved for ginsenoside Re and Rg1 [J. B. Wan et al., J. Pharm. Biomed. Anal., 41 (1), 274-279, 2006].

However, the present invention, for the first time, proves a cost effectiveness and high efficiency process technology for production of Ginsenoside Re constituent in Ginseng leaf extract, excels the performance of currently used C18 silica based HPLC column for analysis of Ginsenoside Re.

it is to understand that while the Gin-Re MIP shown above the invention is intended to illustrate and not to limit the scope of the appended claims. From the technology platform established through Gin-Re imprinted technology, the applications of different kinds of spherical MIP resins imprinted with a template molecule for an individual ginsenoside compound could be used for fast analysis and manufacturing of large scale production of pure ginsenoside compound.

### Example 1

### Separation of Ginsenoside Re and Rg1

Because of their similar molecular structure, analysis of these two compounds in ginseng extracts, biological samples is very challenge. Much effort had been made for better resolution between Rg1 and Re. A representative successful analytical method was reported by Chen et al (Chen Jing, Xu Qun, Jeff Rohrer, Li Lane. LC-GC Asia Pacific, volume 10 (4), 2007). In order to resolve Gin-Re and Gin-Rg1 peaks, the separation was performed in a long gradient and at an elevated temperature of 50° C. The whole analysis time for ginseng extracts was 25 minutes and acetonitrile was used as one component of the mobile phase. Besides the analysis application, none of these methods above were employed for fast purification purpose due to cost and technical factors. However, the analysis of Ginsenoside Re in mixtures or extracts could be significantly accelerated by using the new type column protected by the present invention. Meanwhile, the mobile phase was simplified, only an isocratic mode was needed. Most importantly, within 18 minutes, ginsenoside Re peak was well resolved from other constituents, such as ginsenosides Rg1, Rb1, etc. Therefore, besides ginsenoside Re, this technology could be extended for production of the following major compounds in Panax quinquefolium L. (American ginseng), ginsenosides Rb1, Rb2, Rc, Rd and Rg1 as long as a right template molecule is selected for the individual ginsenoside compound.

In a typical experiment, Gin-Re imprinted spherical polymer beads manufactured by Ginbonds Inc. was used to pack columns. After packed, the columns were conditioned with solvent methanol. The column was used to analyze samples in the following order: methanol blank. ginsenoside Rg1 (2.0 mg/1 mL) and ginsenoside Re (2.0 mg/mL). The column was flushed with methanol between runs. The organic solvents were filtered through a 0.45 μm filter and degassed for removal of dissolved gasses. Samples were dissolved in methanol. The oven temperature was 30° C. and the UV wavelength was set at 203 nm. The data were obtained from an HPLC system consisting of these components as follows: a GS50 Gradient Pump, an AD25 Absorbance Detector, an LC25 Chromatography Oven.

As seen in FIG. 2, Ginsenoside Re was completely separated from ginsenoside Rg1. Other species, such as Ginsenoside Rb1, and 80% total Ginsenoside products were also analyzed, the same results as above were obtained. In these experiments, unlike earlier elution in C18 column separations, Ginsenoside Re was eluted out after Ginsenoside Rg1 or Rb1 on the Surpass™ PolyGin Re column because of much stronger interaction between Ginsenoside Re molecules and MIP polymer beads.

Besides quantitative analyses above, the technology could be used as fast separation of Ginsenoside Re from ginseng extracts for replacement of the currently used processing technologies. To solve high pressure issue, the beads size could be easily tuned to meet the manufacturing purpose. Compared to the currently used conventional processing technologies, this new technology is zero pollution because only methanol is needed for separation, which also can be regenerated easily in laboratories or plants.

Also, the technology platform established here could be used to synthesize Ginsenoside compound-imprinted polymeric beads used for the analysis and manufacturing of the corresponding compound.

What is claimed is:

1. A process technology for producing ultrapure ginsenoside Re compound, Ginsenoside Re could be separated from other ginsenoside compounds in a single step with a simple mobile phase using a key element—Gin-Re imprinted polymeric beads packed column.

2. The mobile phase claimed in claim 1 is methanol only, no buffers or acids or bases were used.

3. The species claimed in claim 1 with molecule structures similar to Ginsenoside Re are ginsenoside Rg1 and Rb1.

4. The organic solvent claimed in claim 1 is methanol and can be recycled easily for future use without quality compromise.
